(19) United States
(12) Patent Application Publication
(10) Pub. No.: US 2008/0234245 A1
(43) Pub. Date: Sep. 25, 2008

(54) Angiostatic Agents for Controlling Choroidal Neovascularisation After Ocular Surgery or Trauma

(76) Inventors: David P. Bingaman, Weatherford, TX (US); Changdong Liu, Grapevine, TX (US); Robert A. Landers, Arlington, TX (US); Xiaolin Gu, Arlington, TX (US)

Correspondence Address: ALCON, IP LEGAL, TB4-8, 6201 SOUTH FREEWAY, FORT WORTH, TX, 76134, US

(21) Appl. No.: 11/568,057

(22) PCT Filed: Apr. 21, 2005
(86) PCT No.: PCT/US05/13653

§ 371 Date: Oct. 18, 2006

Related U.S. Application Data
(60) Provisional application No. 60/564,851, filed on Apr. 23, 2004.

(51) Int. Cl. A61K 31/56; A61P 27/02

(52) U.S. Cl. 514/182

(57) ABSTRACT

Compositions of angiostatic agents for treating choroidal neovascularization resulting from ocular surgery or from trauma to ocular tissue and methods for their use are disclosed.

This application claims priority from U.S. Ser. No. 60/546,815, filed on Apr. 23, 2004.

### BACKGROUND OF THE INVENTION

1. Field of the Invention

This invention is directed to the use of angiostatic agents for treating choroidal neovascularization resulting from surgical procedures.

2. Description of Related Art

Steroids functioning to inhibit angiogenesis in the presence of heparin or specific heparin fragments are disclosed in Crum, et al., A New Class of Steroids Inhibits Angiogenesis In The Presence of Heparin or Heparin Fragment, Science, 230:375-378, Dec. 20, 1985. The authors refer to such steroids as “angiostatic” steroids. Included in the new class of steroids found to be angiostatic are cortisol, cortexolone, and several dihydro and tetrahydro derivatives. In a follow up study directed to testing a hypothesis as to the mechanism by which the steroids inhibit angiogenesis, it was shown that heparin/angiostatic steroid compositions caused dissolution of the basement membrane scaffolding to which anchorage dependent endothelia are attached resulting in capillary involution; see, Ingber, et al. A Possible Mechanism for Inhibition of Angiogenesis by Angiostatic Steroids: Induction of Capillary Basement Membrane Dissolution, Endocrinology 119:1768-1775, 1986.

A group of tetrahydrosteroids useful in inhibiting angiogenesis is disclosed in U.S. Pat. No. 4,975,537, issued to Aristoff, et al. The compounds are disclosed for use in treating head trauma, spinal trauma, septic or traumatic shock, stroke, and hemorrhage shock. In addition, the patent discusses the utility of these compounds in embryo implantation and in the treatment of cancer, arthritis, and arteriosclerosis. The compounds are not disclosed for ophthalmic use. Some of the tetrahydrosteroids disclosed in Aristoff, et al. are disclosed in U.S. Pat. No. 4,771,042 in combination with heparin or a heparin fragment for inhibiting angiogenesis in a warm blooded animal. The patent does not disclose the combination for ophthalmic use.

Compositions of hydrocortisone, “tetrahydrocortisol-S,” and U-72,745G, each in combination with a beta cyclodextrin have been shown to inhibit corneal neovascularization. Li, et al., Angiostatic Steroids Potentiated by Sulphated Cyclodextrin Inhibit Corneal Neovascularization, Investigative Ophthalmology and Visual Science, 32(11):2898-2905, October, 1991. The steroids alone reduce neovascularization somewhat but are not effective alone in providing for regression of neovascularization.

A laser procedure is one method currently used for the inhibition of ocular neovascularization. Photodynamic therapy (PDT) is a procedure in which a photoactivatable dye is given systemically followed by laser activation of the dye in the eye at the site of new blood vessel formation (Asrani & Zeimer, Br J Ophthalmol, 79(8):776-770, August, 1995; Asrani et al, Invest Ophthalmol. Vis Sci, 38(13); 2702-2710, December, 1997; Husain et al, Ophthalmology, 104(8):242-1250, August, 1997; Lin et al, Curr Eye Res, 13(7):513-522, July, 1994.) The photoactivated drug generates free oxygen radicals which seal the newly formed blood vessels. This procedure has been used in patients with the exudative form of macular degeneration and many patients show regression of their subretinal neovascular membranes. Unfortunately, it appears that the PDT induced inhibition of neovascularization is transient lasting only 6-12 weeks (Gragoudas et al, Investigative Ophthalmology & Visual Science, 38(4):S17; Mar. 15, 1997; Sickenberg et al, Investigative Ophthalmology & Visual Science, 38(4):S92, Mar. 15, 1997; Thomas et al, Investigative Ophthalmology & Visual Science, 39(4):S242, Mar. 15, 1998.) There are currently no effective therapies for the treatment of ocular neovascular diseases which do not include the destruction of healthy viable tissue. Although panretinal photocoagulation is the current medical practice for the treatment of diabetic retinopathy and is effective in inhibiting diabetic retinal neovascularization, this procedure destroys healthy peripheral retinal tissue. This destruction of healthy tissue decreases the retinal metabolic demand and thereby reduces retinal ischemia driven neovascularization.

Steroids functioning to inhibit angiogenesis in the presence of heparin or specific heparin fragments are disclosed in Crum, et al., “A New Class of Steroids Inhibits Angiogenesis in the Presence of Heparin or a Heparin Fragment,” Science, 230:1375-1378 (Dec. 20, 1985). The authors refer to such steroids as “angiostatic” steroids. Included within the new class of steroids found to be angiostatic are the dihydro and tetrahydro metabolites of cortisol and cortexolone. In a follow-up study directed to testing a hypothesis as to the mechanism by which the steroids inhibit angiogenesis, it was shown that heparin/angiostatic steroid compositions cause dissolution of the basement membrane scaffolding to which anchorage dependent endothelia are attached resulting in capillary involution; see, Ingber, et al., “A Possible Mechanism for Inhibition of Angiogenesis by Angiostatic Steroids Induction of Capillary Basement Membrane Dissolution,” Endocrinology, 119:1768-1775 (1986).

A group of tetrahydro steroids useful in inhibiting angiogenesis is disclosed in International Patent Application No. PCT/US86/02189, Aristoff, et al., (The Upjohn Company). The compounds are disclosed for use in treating head trauma, spinal trauma, septic or traumatic shock, stroke and hemorrhage shock. In addition, the patent application discusses the utility of these compounds in embryo implantation and in the treatment of cancer, arthritis and arteriosclerosis. The compounds are not disclosed for ophthalmic use.

Tetrahydrocortisol (THF) has been disclosed for its use in lowering the intraocular pressure (IOP) of rabbits made hypertensive with dexamethasone alone, or with dexamethasone/5-beta-dihydrocortisol; see Southren, et al., “Intraocular Hypotensive Effect of a Topically Applied Cortisol Metabolite: 3-alpha, 5-beta-tetrahydrocortisol,” Investigative Ophthalmology and Visual Science, 28 (May, 1987). The authors suggest THF may be useful as an antiglaucoma agent. In U.S. Pat. No. 4,863,912, issued to Southren et al. on Sep. 5, 1989, pharmaceutical compositions containing THF and a method for using these compositions to control intraocular pressure are disclosed. THF has been disclosed as an angiostatic steroid in Folkman, et al., “Angiostatic Steroids,” Ann. Surg., 206(3) (1987) wherein it is suggested angiostatic steroids may have potential use for diseases dominated by abnormal neovascularization, including diabetic retinopathy, neovascular glaucoma and retrolental fibroplasia.

### SUMMARY OF THE INVENTION

Angiostatic steroids and their pharmaceutical formulations are useful for treating choroidal neovascularization resulting from surgical procedures or trauma. The invention is also directed to methods for treating choroidal neovascularization resulting from surgical procedures or trauma using angiostatic steroids.

### BRIEF DESCRIPTION OF THE DRAWINGS

The following drawings form part of the present specification and are included to further demonstrate certain aspects of the present invention. The invention may be better understood by reference to these drawings in combination with the detailed description of specific embodiments presented herein.

FIG. 1 illustrates the proteolytic cascade in angiogenesis and the action of anecortave acetate within the cascade.

FIG. 2 illustrates the proposed mechanism of action of anti-angiogenic agents.

FIGS. 3A, 3B, and 3C. Mouse model of choroidal neovascularization (CNV) induced by rupture of Bruch's membrane. FIG. 3A shows a choroidal flat mount from mouse perfused with fluorescein-labeled dextran at day 14 post-laser (CNV lesions in posterior pole). FIG. 3B shows high magnification of CNV lesion exhibiting focal hyperfluorescence. FIG. 3C shows light micrograph of a fresh frozen retina cross-section stained with GSA lectin at day 14 post-laser. The newly formed vessels and RPE cells extend from the choroid into the subretinal space through the break in bruch's membrane. (Magnification 200×).

FIG. 4A and FIG. 4B. Hyperfluorescent CNV lesions from fluorescein-labeled dextran-stained choroidal flat mounts. FIG. 4A shows the vehicle-treated eye. FIG. 4B shows the eye treated with 10% anecortave acetate. (Digital image, Magnification 200×).

FIG. 5. Graph illustrating that anecortave acetate inhibits laser-induced choroidal neovascularization following a single intravitreal injection in the mouse.

### DETAILED DESCRIPTION OF PREFERRED EMBODIMENTS

Posterior segment neovascularization (NV) is the vision-threatening pathology responsible for the two most common causes of acquired blindness in developed countries: exudative age-related macular degeneration (AMD) and proliferative diabetic retinopathy (PDR). Currently the only approved treatments for posterior segment NV that occurs during exudative AMD is laser photocoagulation or photodynamic therapy with Visudyne®; both therapies involve occlusion of affected vasculature which results in localized laser-induced damage to the retina. For patients with PDR, surgical interventions with vitrectomy and removal of preretinal membranes are the only options currently available. The present invention provides methods for preventing choroidal neovascularization resulting from ocular surgery or trauma to the eye.

Pathologic ocular angiogenesis, which includes posterior segment NV, occurs as a cascade of events that progress from an initiating stimulus to the formation of abnormal new capillaries. The inciting cause in both exudative AMD and PDR is still unknown, however, the elaboration of various proangiogenic growth factors appears to be a common stimulus. Soluble growth factors, such as vascular endothelial growth factor (VEGF), basic fibroblast growth factor (bFGF or FGF-2), insulin-like growth factor 1 (IGF-1), etc., have been found in tissues and fluids removed from patients with pathologic ocular angiogenesis. Following initiation of the angiogenic cascade, the capillary basement membrane and extracellular matrix are degraded and capillary endothelial cell proliferation and migration occur. Endothelial sprouts anastomose to form tubes with subsequent patent lumen formation. The new capillaries commonly have increased vascular permeability or leakiness due to immature barrier function, which can lead to tissue edema. Differentiation into a mature capillary is indicated by the presence of a continuous basement membrane and normal endothelial junctions between other endothelial cells and pericytes; however, this differentiation process is often impaired during pathologic conditions.

Effective treatment of the choroidal neovascularization, pathologic ocular angiogenesis and edema, which can result from ocular surgery or other traumas to the ocular tissue, would improve the patient's quality of life and productivity within society. Also, societal costs associated with providing assistance and health care to the blind could be dramatically reduced.

The development of blood vessels for the purpose of sustaining viable tissue is known as angiogenesis. Agents which inhibit angiogenesis are known by a variety of terms such as angiostatic, angiolytic or angiotropic agents. For purposes of this specification, the term “angiostatic agent” means compounds which can be used to inhibit angiogenesis.

The specific angiostatic agents of the present invention are steroids or steroid metabolites. For purposes herein, the term “angiostatic steroids” means steroids and steroid metabolites which inhibit angiogenesis. The present invention is based on the finding that angiostatic steroids can be used for the treatment of choroidal neovascularization, and other conditions, resulting from ocular surgery or trauma to ocular tissues.

Preferred angiostatic steroids of the present invention have the following formula:

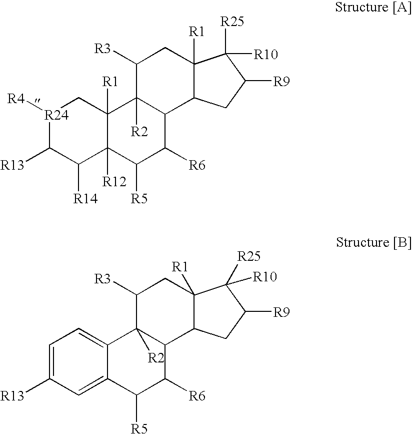

wherein R₁ is H, β-CH₃ or β-C₂H₅;
R₂ is F, C₉-C₁₁ double bond, C₉-C₁₁ epoxy, H or Cl;
R₃ is H, OR₂₆, OC(═O)R₂₇, halogen, C₉-C₁₁ double bond, C₉-C₁₁ epoxy, ═O, —OH, —O-alkyl(C₁-C₁₂),

### —OC(═O)alkyl(C₁-C₁₂), —OC(═O)ARYL, —OC(═O)N(R)₂ or

—OC(═O)OR₇, wherein ARYL is furyl, thienyl, pyrrolyl, or pyridyl and each of said moieties is optionally substituted with one or two (C₁-C₄)alkyl groups, or ARYL is —(CH₂)_{f}-phenyl wherein f is 0 to 2 and the phenyl ring is optionally substituted with 1 to 3 groups selected from chlorine, fluorine, bromine, alkyl(C₁-C₃), alkoxy(C₁-C₃), thioalkoxy-(C₁-C₃), Cl₃C—, F₃C—, —NH₂ and —NHCOCH₃ and R is hydrogen, alkyl (C₁-C₄), or phenyl and each R can be the same or different, and R₇ is ARYL as herein defined, or alkyl(C₁-C₁₂);

### R₄ is H, CH₃, Cl or F;

R₅ is H, OH, F, Cl, Br, CH₃, phenyl, vinyl or allyl;

### R₆ is H or CH₃;

R₉ is CH₂CH₂OR₂₆, CH₂CH₂OC(═O)R₂₇, H, OH, CH₃, F, ═CH₂, CH₂C(═O)OR₂₈, OR₂₆, O(C═O)R₂₇ or O(C═O)CH₂(C═O)OR₂₆
R₁₀ is —C≡CH, —CH═CH₂, halogen, CN, N₃, OR₂₆, OC(═O)R₂₇, H, OH, CH₃ or R₁₀ forms a second bond between positions C-16 and C-17;
R₁₂ is H or forms a double bond with R₁ or R₁₄;
R₁₃ is halogen, OR₂₆, OC(═O)R₂₇, NH₂, NHR₂₆, NHC(═O)R₂₇, N(R₂₆)₂, NC(═O)R₂₇, N₃, H, —OH, ═O, —O—P(═O)(OH)₂, or —O—C(═O)—(CH₂)ₜCOOH where t is an integer from 2 to 6;
R₁₄ is H or forms a double bond with R₁₂;

### R₁₅ is H, ═O or —OH;

and R₂₃ with R₁₀ forms a cyclic phosphate;
wherein R₉ and R₁₅ have the meaning defined above;
or wherein R₂₃ is —OH, O—C(═O)—R₁₁, —OP(O)—(OH)₂, or —O—C(═O)—(CH₂)ₜCOOH wherein t is an integer from 2 to 6; and R₁₁ is —Y—(CH₂)ₙ—X—(CH₂)ₘ—SO₃H, —Y′—(CH₂)ₚ—X′—(CH₂)_{q}—NR₁₆R₁₇ or -Z(CH₂)ᵣQ,
wherein Y is a bond or —O—; Y′ is a bond, —O—, or —S—; each of X and X′ is a bond, —CON(R₁₈)—, —N(R₁₈)CO—, —O—, —S—, —S(O)—, or —S(O₂)—; R₁₈ is hydrogen or alkyl (C₁-C₄);
each of R₁₆ and R₁₇ is a lower alkyl group of from 1 to 4 carbon atoms optionally substituted with one hydroxyl or R₁₆ and R₁₇ taken together with the nitrogen atom to which each is attached forms a monocyclic heterocycle selected from pyrrolidino, piperidino, morpholino, thiomorpholino, piperazino or N(lower)alkyl-piperazino wherein alkyl has from 1 to 4 carbon atoms; n is an integer of from 4 to 9; m is an integer of from 1 to 5; p is an integer of from 2 to 9; q is an integer of from 1 to 5;
Z is a bond or —O—; r is an integer of from 2 to 9; and Q is one of the following:

(1) —R₁₉—CH₂COOH wherein R₁₉ is —S—, —S(O)—, —S(O)₂—, —SO₂N(R₂₀)—, or N(R₂₀)SO₂—; and R₂₀ is hydrogen or lower alkyl-(C₁-C₄); with the proviso that the total number of carbon atoms in R₂₀ and (CH₂)ᵣ is not greater than 10; or

(2) —CO—COOH; or

(3) CON(R₂₁)CH(R₂₂)COOH wherein R₂₁ is H and R₂₂ is H, CH₃, —CH₂COOH, —CH₂CH₂COOH, —CH₂OH, —CH₂SH, —CH₂CH₂SCH₃, or

—CH₂Ph-OH wherein Ph-OH is p-hydroxyphenyl;

### or R₂₁ is CH₃ and R₂₂ is H;

or R₂₁ and R₂₂ taken together are —CH₂CH₂CH₂—;
or —N(R₂₁)CH(R₂₂)COOH taken together is —NHCH₂CONHCH₂COOH; and pharmaceutically acceptable salts thereof;
with the proviso that if R₂₃ is a phosphate, it must form a cyclic phosphate, with R₁₀ when R₁₃ is ═O, except for the compound wherein R₁ is β-CH₃, R₂ and R₃ taken together form a double bond between positions 9 and 11, R₄ and R₆ are hydrogen, R₁₂ and R₁₄ taken together form a double bond between positions 4 and 5, R₅ is α-F, R₉ is β-CH₃, R₁₀ is α-OH, R₁₃ and R₁₅ are ═O and R₂₃ is —OP(O)—(OH)₂.
R₂₄═C, C₁-C₂ double bond, O;
R₂₅═C(R₁₅)CH₂—R₂₃, OH, OR₂₆, OC(═O)R₂₇, R₂₆, COOH, C(═O)OR₂₆, CHOHCH₂OH, CHOHCH₂OR₂₆, CHOHCH₂C(═O)R₂₇, CH₂CH₂OH, CH₂CH₂OR₂₆, CH₂CH₂OC(═O)R₂₇, CH₂CN, CH₂N₃, CH₂NH₂, CH₂NHR₂₆, CH₂N(R₂₆)₂, CH₂OH, CH₂OR₂₆, CH₂O(C═O)R₂₇, CH₂O(P═O) (OH)₂, CH₂O(P═O)(OR₂₆)₂, CH₂SH, CH₂S—R₂₆, CH₂SC(═O)R₂₇, CH₂NC(═O)R₂₇, C(═O)CHR₂₈OH, C(═O)CHR₂₈OR₂₆, C(═O)CHR₂₈OC(═O)R₂₇ or
R₁₀ and R₂₅ taken together may be ═C(R₂₈)₂, that is, an optionally alkyl substituted methylene group;
wherein R₂₆═C₁-C₆ (alkyl, branched alkyl, cycloalkyl, haloalkyl, aralkyl, aryl);
R₂₇=R₂₆+OR₂₆; R₂₈=H, C₁-C₆ (alkyl, branched alkyl, cycloalkyl).

Unless specified otherwise, all substituent groups attached to the cyclopentanophenanthrene moiety of Structures [A] and [B] may be in either the alpha or beta position. Additionally, the above structures include all pharmaceutically acceptable salts of the angiostatic steroids.

Preferred angiostatic steroids are 21-methyl-5β-pregnan-3α,11β,17α,21-tetrol-20-one 21-methyl ether; 3β-azido-5β-pregnan-11β,17α,21-triol-20-one-21-acetate; 3β-azido-21-acetoxy-5β-pregnan-11β,17α-diol-20-one; 3β-acetamido-21-acetoxy-5β-pregnan-11β,17α-diol-20-one; 3β-acetamido-21-acetoxy-5β-pregnan-11β,17α-diol-20-one acetate; 5β-pregnan-11β,17α,21-triol-20-one; 17-((4-fluoro)thiophenoxy)methyl-1,3,5-estratrien-3,17-diol; 20-azido-21-nor-5β-pregnan-3α,17α-diol; 20-(carbethoxymethyl)thio-21-nor-5β-pregnan-3α,17α-diol; 20-(4-fluorophenyl)thio-21-nor 5β-pregnan-3α,17β-diol; 20-acetamido-21-nor-5β-pregnan-17α-diol-3-acetate; 16α-(2-hydroxyethyl)-17β-methyl-5β-androstan-3α,17β-diol; 20-cyano-21-nor-5β-pregnan-3α,17α-diol; 17α-methyl-5β-androstan-3α,17β-diol; 21-nor-5β-pregn-17(20)-en-3α-ol; 21-nor-5β-pregn-17(20)-en-3α-ol-3-acetate; 21-nor-5β-pregn-17(20)-en-3α-ol-16-acetic acid-3-acetate; 21-nor-5β-pregnan-3α,17α,20-triol; 21-nor-5β-pregnan-3α,17α,20-triol-3-acetate; 4, 9(11)-pregnadien-17α,21-diol-3,20-dione-21-acetate and 4,9(11)-pregnadien-17α,21-diol-3,20-dione.

The more preferred compounds are 21-methyl-5β-pregnan-3α,11β,17α,21-tetrol 20-one-21-methyl ether; 3β-azido-21-acetoxy-5β-pregnan-11β,17α-diol-20-one; 3β-acetamido-21-acetoxy-5β-pregnan-11β,17α-diol-20-one; and 5β-pregnan-11β,17α,21-triol-20-one. The most preferred compounds are 4,9(11)-pregnadien-17α,21-diol-3,20-dione-21-acetate (anecortave acetate) and 4,9(11)-pregnadien-17α,21-diol-3,20-dione.

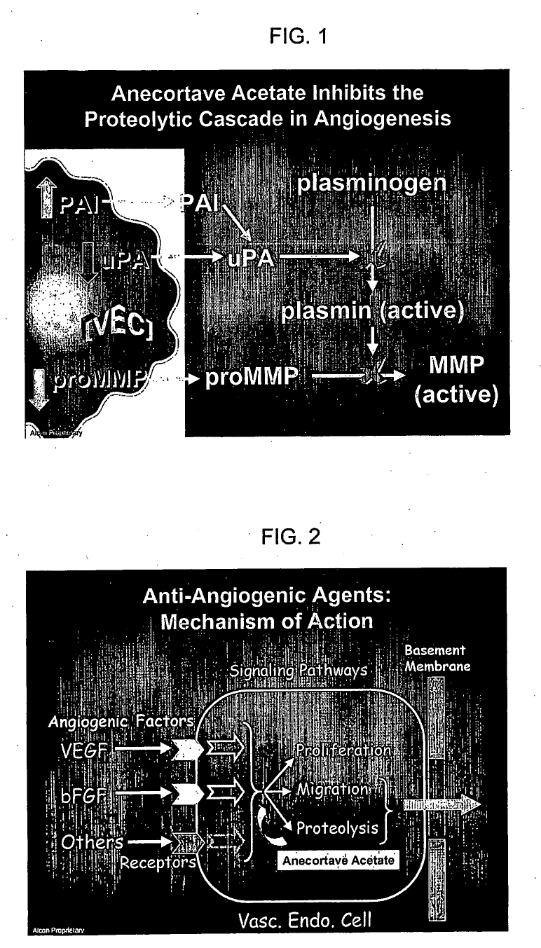

Anecortave acetate represents a new antiangiogenic class, cortisenes, that inhibit pathologic ocular angiogenesis. (Clark A F, EXP. OPIN. INVEST. DRUGS 12:1867-1877 (1999); Benezra D. et al., INVEST. OPHTHALMOL. VIS. SCI. 38:1954-1962 (1997); Clark A F, et al., INVEST. OPHTHALMOL. VIS. SCI. 40:2156-2162 (1999); DeFaller J M, Clark A F. In: PTERYGIUM. Kugler Publ., The Hague, Netherlands, 2000; Penn J S, et al., INVEST OPHTALMOL. VIS. SCI. 42:283-290 (2001)). Structurally derived from a steroid hormone backbone, permanent chemical modifications have rendered the compound devoid of conventional glucocorticoid (anti-inflammatory) or mineralocorticoid activity, while maintaining its potent antiangiogenic activity. (Clark 1999). Anecortave acetate provides reproducible efficacy against posterior segment neovascularization (NV), as evidenced by its published activity following intravitreal injection in the rat OIR model. (Penn 2001). The antiangiogenic effects of anecortave acetate have been attributed, in part, to a decrease in the expression of uPA (urokinase-type plasminogen activator) and matrix metalloproteinases, and an increase in the expression of PAI-1 (plasminogen activator inhibitor) (FIG. 1 and FIG. 2) (DeFaller and Clark 2000; Penn 2001).

The angiostatic steroids of the present invention may be incorporated in various formulations for delivery to the eye. For example, topical formulations can be used and can include opthalmologically acceptable preservatives, surfactants, viscosity enhancers, buffers, sodium chloride and water to form aqueous sterile ophthalmic solutions and suspensions. In order to prepare sterile ophthalmic ointment formulations, an angiostatic steroid is combined with a preservative in an appropriate vehicle, such as mineral oil, liquid lanolin or white petrolatum. Sterile ophthalmic gel formulations comprising the angiostatic steroids of the present invention can be prepared by suspending an angiostatic steroid in a hydrophilic base prepared from a combination of, for example, Carbopol-940 (a carboxyvinyl polymer available from the B.F. Goodrich Company) according to published formulations for analogous ophthalmic preparations. Preservatives and tonicity agents may also be incorporated in such gel formulations.

The specific type of formulations selected will depend on various factors, such as the angiostatic steroid or its salt being used, and the dosage frequency. Topical ophthalmic aqueous solutions, suspensions, ointments and gels are the preferred dosage forms. The angiostatic steroid will normally be contained in these formulations in an amount of from about 0.005 to about 5.0 weight percent (wt. %). Preferable concentrations range from about 0.05 to about 2.0 wt. %. Thus, for topical administration, these formulations are delivered to the surface of the eye one to four times per day, depending upon the routine discretion of the skilled clinician.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

| Component | wt. % |
|---|---|
| Angiostatic Steroid | 0.005-5.0 |
| Tyloxapol | 0.01-0.05 |
| HPMC | 0.5 |
| Benzalkonium Chloride | 0.01 |
| Sodium Chloride | 0.8 |
| Edetate Disodium | 0.01 |
| NaOH/HCl | q.s. pH 7.4 |
| Purified Water | q.s. 100 mL |

### EXAMPLE 2

| Component | wt. % |
|---|---|
| 4,9(11)-pregnadien-17α,21-diol-3,20-dione-21-acetate | 1.0 |
| Mannitol | 2.40 |
| Carbopol 974P | 0.50 |
| Polysorbate 80 | 0.05 |
| Benzalkonium Chloride | 0.01 |
| Sodium Chloride | 0.4 |
| Edetate Disodium | 0.01 |
| NaOH/HCl | q.s. pH 7.4 |
| Purified Water | q.s. 100 mL |

### EXAMPLE 3

### Preparation of 5β-Pregnan-11β,17α,21-triol-20-one

Tetrahydrocortisol-F-21-t-butyldiphenylsilyl Ether (ES03842)

A solution of 4.75 g (17.3 mmol) of t-butyldiphenylchlorosilane in 5 mL of dry DMF was added dropwise to a stirred solution of 5.7 g (15.6 mmol) of tetrahydrocortisol-F (Steraloids No. P9050) and 2.3 g (19 mmol) of 4-dimethylaminopyridine (DMAP) in 30 mL of dry DMF, under N₂, at −25 to −30° C. (maintained with CO₂—MeCN). After a further 20 min at −30° C., the mixture was allowed to warn to 23° C. overnight.

The mixture was partitioned between ether and water, and the organic solution was washed with brine, dried (MgSO₄), filtered and concentrated to give 10.7 g of a white foam.

This material was purified by flash column chromatography (400 g silica; 62.5 to 70% ether/hexane). The 3-siloxy isomer eluted first, followed by mixed fractions, followed by the title compound. The concentrated mixed fractions (4.0 g) were chromatographed on the same column with 35% ethyl acetate/hexane. The total yield of the 3-siloxy isomer was 0.42 g (5%), and of the title compound, 5.05 g (53.5%). Continued elution with 25% MeOH/EtOAc allowed recovery of unreacted tetrahydrocortisol-F.

### PSO3842

NMR (200 MHz ¹H) (CDCl₃): δ 0.63 (s, 3H, Me-18); 1.11 (s, 9H, t-Bu); 1.12 (s, 3H, Me-19); 2.57 (t, J=13, 1H, H-8); 2.6 (s, 1H, OH-17); 3.63 (sept, J=2.5, 1H, H-3); 4.15 (br s, 1H, H-11); 4.37 and 4.75 (AB, J=20, 2H, H-21); 7.4 (m, 6H) and 7.7 (m, 4H) (Ph₂).

NMR (200 MHz ¹H) (DMSO-d₆): δ 0.64 (s, 3H, Me-18); 1.02 (s, 9H, t-Bu); 1.07 (s, 3H, Me-19); 2.50 (t, J=13, 1H, H-8); 3.37 (m, 1H, H-3); 3.94 (d, J=2, 1H, OH-11); 4.00 (br s, 1H, H-11); 4.42 (d, J=5, 1H, OH-3); 4.38 and 4.83 (AB, J=20, 2H, H-21); 5.11 (s, 1H, OH-17); 7.45 (m, 6H) and 7.6 (m, 4H) (Ph₂).

NMR (50.3-MHz ¹³C) (CDCl₃): 17.4 (C-18); 19.3 (C-16); 23.7 (C-15); 26.3 (C-7); 26.6 (C-19); 26.8 (Me₃C); 27.2 (C-6); 30.9 (C-2); 31.5 (C-8); 34.1 (Me₃C); 34.8 (C-10); 35.2 (C-1); 36.2 (C-4); 39.7 (C-13); 43.5 (C-5); 44.3 (C-9); 47.4 (C-12); 52.1 (C-14); 67.8 (C-11); 68.9 (C-21); 71.7 (C-3); 89.8 (C-14); 127.8, 129.8, 132.8, 132.9, 135.7, 135.8 (diastereotopic Ph₂); 208.8 (C-20). Underlined resonances showed inversion in the APT experiment. Assignments: E. Breitmaier, W. Voelter “Carbon-13 NMR Spectroscopy,” 3d ed., VCH, 1987; pp. 345-348.

IR (KBr) 3460, 2930, 2860, 1720, 1428, 1136, 1113, 1070, 1039, 703 cm⁻¹.

This compound did not show a sharp melting point but turned to a foam at 80-100° C. Numerous attempts at recrystallization failed.

### 5β-Pregnan-11β,17α,21-triol-20-one

A solution of PSO3842 (0.91 g, 1.50 mmol) and thiocarbonyl diimidazole (1.05 g, 5.9 mmol) in 8 mL of anhydrous dioxane was refluxed under N₂ for 3.5 h. The cooled solution was partitioned between ether and water and the organic solution was washed with brine, dried (MgSO₄), filtered and concentrated. The residue was chromatographed (120 g SiO₂, 35% EtOAc/hexane) giving 0.86 g (80%) of the imidazolyl thioester.

A solution of 0.75 g (1.05 mmol) of this compound in 100 mL of anhydrous dioxane was added dropwise over 2.2 h to a rapidly stirred, refluxing solution of 1.6 mL (5.9 mmol) of Bu₃SnH in 100 mL of anhydrous dioxane under N₂. After a further 1 h at reflux, the solution was cooled, concentrated and the residue chromatographed (200 g SiO₂, 9% EtOAc/hexane) giving 0.43 g (70%) of the 3-deoxy-21-silyl ether. This material was dissolved in 20 mL of methanol; Bu₄NF.3H₂O (0.50 g, 1.6 mmol) was added, and the mixture was heated to reflux under N₂ for 4 h. The cooled solution was diluted with 2 volumes of EtOAc, concentrated to ¼ volume, partitioned (EtOAc/H₂O), and the organic solution was washed with brine, dried (MgSO₄), filtered and concentrated. The residue (0.40 g) was chromatographed (30 g SiO₂, 40% EtOAc/hexane) to give 0.25 g (98%) of an oil.

This oil was crystallized (n-BuCl) to afford 0.14 g of the title compound as a white solid, m.p. 167-170° C.

IR (KBr): 3413 (br), 2934, 1714, 1455, 1389, 1095, 1035 cm⁻¹.

MS (CI): 351 (M+1).

NMR (200 MHz ¹H, DMSO-d₆): δ 0.69 (s, 3H, Me-18); 1.14 (s, 3H, Me-19); 0.8-2.0 (m); 2.5 (t, J=13, 1H, H-8); 3.96 (d, J=2, 1H, OH-11); 4.1 (br s, 1H, H-11); 4.1 and 4.5 (AB, further split by 5 Hz, 2H, H-21); 4.6 (t, J=5, 1H, OH-21); 5.14 (s, 1H, OH-17).

Anal. Calc'd for C₂₁H₃₄O₄: C, 71.96; H, 9.78.

Found: C, 71.69; H, 9.66.

### EXAMPLE 4

### Preparation of 21-Methyl-5β-pregnan-3α,11β,17α,21-tetrol-20-one 21-methyl ether

Sodium hydride (60% oil dispersion, 0.10 g, 2.5 mmol) was added to a stirred solution of tetrahydrocortisol-F (0.73 g, 2.0 mmol) and CH₃I (0.60 mL, 9.6 mmol) in 8 mL of anhydrous DMF under N₂. Hydrogen was evolved, and the temperature rose to 35° C. After 1 h, the mixture was diluted with EtOAc, extracted with water (until neutral) and brine, dried (MgSO₄), filtered and concentrated. The residue was chromatographed (70 g SiO₂, 80% EtOAc/hexane) to give 0.17 g of a white solid, MS (CI)=395 (M+1). This material was recrystallized (EtOAc-n-BuCl) to afford 0.12 g (16%) of the title compound as a feathery white solid, m.p. 208-213° C.

IR (KBr): 3530, 3452, 2939, 2868, 1696 (s, CO), 1456, 1366, 1049 cm⁻¹.

NMR (200 MHz ¹H, DMSO-d₆): δ 0.74 (s, 3H, Me-18); 1.09 (s, 3H, Me-19); 1.14 (d, J=6.6, 3H, C-21 Me); 0.8-2.0 (m); 2.47 (t, J=13, 1H, H-8); 3.18 (s, 3H, OMe); 3.35 (m, 1H, H-3); 4.00 (d, J=2, 1H, OH-11); 4.07 (br s, 1H, H-11); 4.37 (q, J=6.6, 1H, H-21); 4.43 (d, J=5, 1H, OH-3); 5.16 (s, 1H, OH-17).

Anal. Calc'd for C₂₃H₃₈O₅: C, 70.01; H, 9.71.

Found: C, 70.06; H, 9.76.

### EXAMPLE 5

### Preparation of 3β-Azido-21-acetoxy-5β-pregnan-11β, 17α-diol-20-one

A solution of triphenylphosphine (2.6 g, 10 mmol) in 10 mL of toluene was carefully added to a stirred solution of PSO3842 (see Example 4) (1.75 g, 2.90 mmol), diphenylphosphoryl azide (2.2 mL, 10.2 mmol) and diethyl azodicarboxylate (1.55 mL, 10 mmol) under N₂, keeping the internal temperature below 35° C. (exothermic). The solution was stirred for 1.2 h, then diluted with ether, washed with water and brine, dried (MgSO₄), filtered and concentrated and the residue (9.5 g, oil) chromatographed 175 g SiO₂, 15% EtOAc/hexane) giving 1.83 g of a viscous oil.

A solution of 1.73 g of this material and 1.75 g (5.5 mmol) of Bu₄NF.3H₂O in 20 mL of methanol was refluxed under N₂ for 2.5 h. The crude product (1.94 g) was isolated with ethyl acetate and chromatographed (100 g SiO₂, 50% EtOAc/hexane) giving 0.60 g (56%) of a white semisolid. Trituration (4:1 hexane-ether) gave 0.57 g (53%) of a solid.

A stirred solution of 0.40 g of this material in 3 mL of dry pyridine was treated with 0.3 mL of acetic anhydride and stirred overnight at 23° C. under N₂. The mixture was quenched with 1 mL of methanol, stirred for 15 min, diluted with ether, washed with 1 M aqueous HCl, water (until neutral), brine, dried (MgSO4), filtered and concentrated. The residue (0.41 g, oil) was chromatographed (35 g SiO₂, 33% EtOAc/hexane) to afford 0.33 g (76%) of the title compound as a white foam, m.p. 80-90° C. (dec).

IR (KBr): 3505, 2927, 2866, 2103 (vs), 1721 (sh 1730), 1268, 1235 cm⁻¹.

NMR (200 MHz ¹H, CDCl₃): δ 0.92 (s, 3H, Me-18); 1.21 (s, 3H, Me-19); 1.0-2.1 (m); 2.17 (s, 3H, Ac); 2.25 (s 1H, OH-17); 2.74 (m, 1H, H-8); 3.97 (br s, 1H, H-3); 4.31 (br s, 1H, H-11); 4.94 (AB, J=17, Δv=60, 2H, H-21).

Anal. Calc'd for C₂₃H₃₅N₃0₅: C, 63.72; H, 8.14; N, 9.69.

Found: C, 63.39; H, 8.18; N, 9.45.

### EXAMPLE 6

### Preparation of 3β-Acetamido-21-acetoxy-5β-pregnan-11β, 17α-diol-20-one

A solution of 3β-azido-21-acetoxy-5β-pregnan-11β,17α-diol-20-one (0.15 g, 0.35 mmol) in 8 mL of absolute ethanol containing 0.03 g of 10% Pd on C was stirred under H₂ (1 atm) at 23° C. for 2 h. The mixture was filtered and concentrated, the residue dissolved in EtOAc, the basic material extracted into 1 M aqueous HCl, liberated (Na₂CO₃), extracted (EtOAc) and the organic extract washed with water (until neutral) and brine, dried (MgSO₄), filtered and concentrated to provide 58 mg of a solid.

This material was acetylated (1.0 mL of dry pyridine, 0.20 mL of Ac₂O, 23° C., N², overnight), followed by workup (as described for the steroid of Example 6 [last step]) affording a crude product that was chromatographed (25 g SiO₂, EtOAc). This product was triturated with ether to afford 51 mg (33%) of product as a white solid, m.p. 179-181° C.

Ms (CI, isobutane): (M+1)=450 (M⁺), 432, 391, 371, 348.

IR (KBr): 3398 (br), 2932, 2865, 1720 (sh. 1740), 1652, 1538, 1375, 1265, 1236 cm⁻¹.

NMR (200 MHz ¹H, CDCl₃): δ 0.89, 1.22, 1.99, 2.17 (all s, 3H); 1.0-2.2 (m); 2.7 (t, J=13, 1H, H-8); 3.03 (s, 1H, OH-17); 4.2 (br s, 1H, H-11); 4.3 (br s, 1H, H-3); 4.96 (AB, J=17.5, Δv=42, 2H, H-21); 5.8 (d, J=10, 1H, NH).

### EXAMPLE 7

### Treatment of Choroidal Neovascularization Induced in Mice

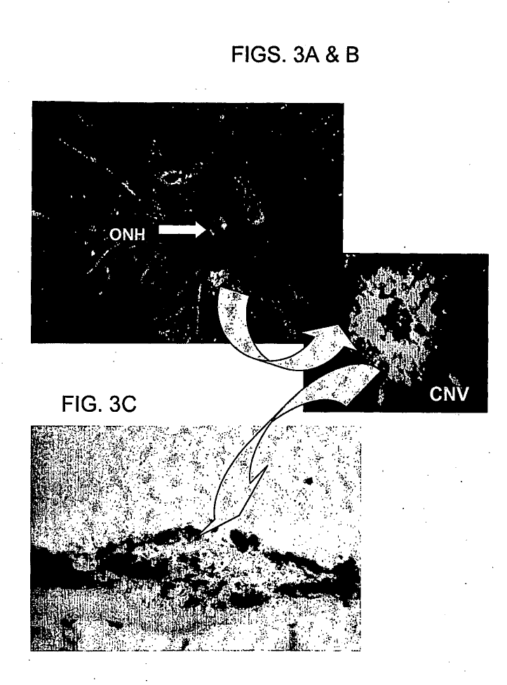

Choroidal neovascularization (CNV) was induced in C57BL/J mice by rupturing Bruch's membrane via focal laser photocoagulation (FIG. 3). (Tobe T, et al., AM. J. PATHOL., 153:1641-1646 (1998)). Three to four retinal burns were placed in randomly assigned eye using the Alcon 532 nm EyeLite laser (75 μm spot size, 0.1 seconds duration, 120 mW) with a slit lamp delivery system. The laser burns were used to generate a rupture in Bruch's membrane, which was indicated opthalmoscopically by the formation of a bubble under the retina. Only mice with laser burns that produced three bubbles per eye were included in the study. Burns were typically placed at the 3, 6, 9 or 12 o'clock positions in the posterior pole of the retina, avoiding the branch retinal arteries and veins.

Each mouse was randomly assigned into one of the following treatment groups after laser: noninjected controls, sham-injected controls, vehicle-injected mice, or one of three anecortave acetate-injected groups. Control mice received laser photocoagulation in both eyes, where one eye received a sham injection, i.e. a pars plana needle puncture. For intravitreal-injected animals, one laser-treated eye received a 5 μl intravitreal injection of 0%, 0.1%, 1%, or 10% anecortave acetate. The intravitreal injection was performed immediately after laser photocoagulation.

Fourteen days post-laser, all mice were euthanized and systemically perfused with fluorescein-labeled dextran. Eyes were then harvested and prepared as choroidal flat mounts, and the 2-dimensional CNV area was quantified with computerized digital analysis, (Mori K., et al., AM. J. PATHOL. 159:313-320 (2001)). The median CNV area/burn per mouse per treatment was used for statistical analysis; P≦0.05 was considered significant.

Results:

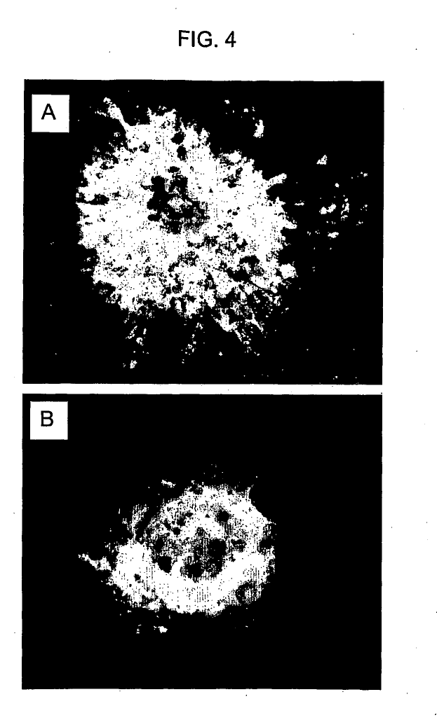

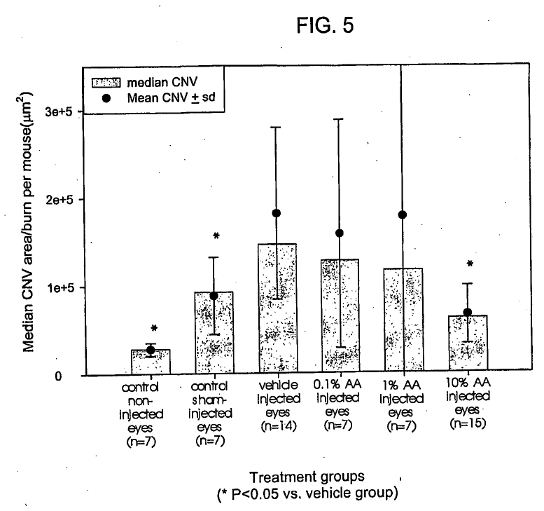

A gross reduction in CNV development was observed as a decrease in the hyperfluorescent area at the site of laser photocoagulation in eyes injected with 10% anecortave acetate versus controls. (FIG. 4). An overall significant difference between treatment groups was established with a Kruskal-Wallis one way ANOVA (P<0.001) (FIG. 5). Eyes injected with 10% anecortave acetate exhibited significant inhibition of CNV (↓57.8%) as compared to vehicle-injected eyes (Mann-Whitney rank sum test; P<0.001). No difference was observed between eyes injected with 0.1% or 1% anecortave acetate and vehicle-injected controls.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

All references cited herein, to the extent that they provide exemplary procedural or other details supplementary to those set forth herein, are specifically incorporated herein by reference.

We claim:

1. A method for treating choroidal neovascularization resulting from ocular surgery or trauma to ocular tissue, said method comprising administering a pharmaceutically effective amount of an angiostatic agent having the following structure: 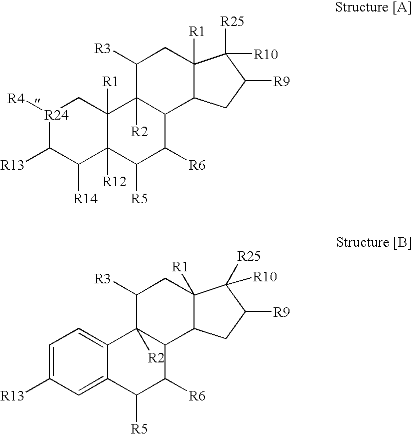
wherein R₁ is H, β-CH₃ or β-C₂H₅;
R₂ is F, C₉-C₁₁ double bond, C₉-C₁₁ epoxy, H or Cl;
R₃ is H, OR₂₆, OC(═O)R₂₇, halogen, C₉-C₁₁ double bond, C₉-C₁₁ epoxy, ═O, —OH, —O-alkyl(C₁-C₁₂),
—OC(═O)alkyl(C₁-C₁₂), —OC(═O)ARYL, —OC(═O)N(R)₂ or
—OC(═O)OR₇, wherein ARYL is furyl, thienyl, pyrrolyl, or pyridyl and each of said moieties is optionally substituted with one or two (C₁-C₄)alkyl groups, or ARYL is —(CH₂)_{f}-phenyl wherein f is 0 to 2 and the phenyl ring is optionally substituted with 1 to 3 groups selected from chlorine, fluorine, bromine, alkyl(C₁-C₃), alkoxy(C₁-C₃), thioalkoxy-(C₁-C₃), Cl₃C—, F₃C—, —NH₂ and —NHCOCH₃ and R is hydrogen, alkyl (C₁-C₄), or phenyl and each R can be the same or different, and R₇ is ARYL as herein defined, or alkyl(C₁-C₁₂);
R₄ is H, CH₃, Cl or F;
R₅ is H, OH, F, Cl, Br, CH₃, phenyl, vinyl or allyl;
R₆ is H or CH₃;
R₉ is CH₂CH₂OR₂₆, CH₂CH₂C(═O)R₂₇, H, OH, CH₃, F, ═CH₂, CH₂C(═O)OR₂₈, OR₂₆, O(C═O)R₂₇ or O(C═O)CH₂(C═O)OR₂₆
R₁₀ is —C≡CH, —CH═CH₂, halogen, CN, N₃, OR₂₆, OC(═O)R₂₇, H, OH, CH₃ or R₁₀ forms a second bond between positions C-16 and C-17;
R₁₂ is H or forms a double bond with R₁ or R₁₄;
R₁₃ is halogen, OR₂₆, OC(═O)R₂₇, NH₂, NHR₂₆, NHC(═O)R₂₇, N(R₂₆)₂, NC(═O)R₂₇, N₃, H, —OH, ═O, —O—P(═O)(OH)₂, or —O—C(═O)—(CH₂)ₜCOOH where t is an integer from 2 to 6;
R₁₄ is H or forms a double bond with R₁₂;
R₁₅ is H, ═O or —OH;
and R₂₃ with R₁₀ forms a cyclic phosphate;
wherein R₉ and R₁₅ have the meaning defined above;
or wherein R₂₃ is —OH, O—C(═O)—R₁₁, —OP(O)—(OH)₂, or —O—C(═O)—(CH₂)COOH wherein t is an integer from 2 to 6; and R₁₁ is —Y—(CH₂)ₙ—X—(CH₂)ₘ—SO₃H, —Y′—(CH₂)ₚ—X′—(CH₂)_{q}—NR₁₆R₁₇ or -Z(CH₂)ᵣQ,
wherein Y is a bond or —O—; Y′ is a bond, —O—, or —S—; each of X and X′ is a bond, —CON(R₁₈)—, —N(R₁₈)CO—, —O—, —S—, —S(O)—, or —S(O₂)—; R₁₈ is hydrogen or alkyl (C₁-C₄);
each of R₁₆ and R₁₇ is a lower alkyl group of from 1 to 4 carbon atoms optionally substituted with one hydroxyl or R₁₆ and R₁₇ taken together with the nitrogen atom to which each is attached forms a monocyclic heterocycle selected from pyrrolidino, piperidino, morpholino, thiomorpholino, piperazino or N(lower)alkyl-piperazino wherein alkyl has from 1 to 4 carbon atoms; n is an integer of from 4 to 9; m is an integer of from 1 to 5; p is an integer of from 2 to 9; q is an integer of from 1 to 5;
Z is a bond or —O—; r is an integer of from 2 to 9; and Q is one of the following:
(1) —R₁₉—CH₂COOH wherein R₁₉ is —S—, —S(O)—, —S(O)₂—, —SO₂N(R₂₀)—, or N(R₂₀)SO₂—; and R₂₀ is hydrogen or lower alkyl-(C₁-C₄); with the proviso that the total number of carbon atoms in R₂₀ and (CH₂)ᵣ is not greater than 10; or
(2) —CO—COOH; or
(3) CON(R₂₁)CH(R₂₂)COOH wherein R₂, is H and R₂₂ is H, CH₃, —CH₂COOH, —CH₂CH₂COOH, —CH₂OH, —CH₂SH, —CH₂CH₂SCH₃, or
—CH₂Ph-OH wherein Ph-OH is p-hydroxyphenyl;
or R₂₁ is CH₃ and R₂₂ is H;
or R₂₁ and R₂₂ taken together are —CH₂CH₂CH₂—;
or —N(R₂₁)CH(R₂₂)COOH taken together is —NHCH₂CONHCH₂COOH; and
pharmaceutically acceptable salts thereof;
with the proviso that if R₂₃ is a phosphate, it must form a cyclic phosphate, with R₁₀ when R₁₃ is ═O, except for the compound wherein R₁ is β-CH₃, R₂ and R₃ taken together form a double bond between positions 9 and 11, R₄ and R₆ are hydrogen, R₁₂ and R₁₄ taken together form a double bond between positions 4 and 5, R₅ is α-F, R₉ is β-CH₃, R₁₀ is α-OH, R₁₃ and R₁₅ are ═O and R₂₃ is —OP(O)—(OH)₂.
R₂₄═C, C₁-C₂ double bond, O;
R₂₅═C(R₁₅)CH₂—R₂₃, OH, OR₂₆, OC(═O)R₂₇, R₂₆, COOH, C(═O)OR₂₆, CHOHCH₂OH, CHOHCH₂OR₂₆, CHOHCH₂C(═O)R₂₇, CH₂CH₂OH, CH₂CH₂OR₂₆, CH₂CH₂C(═O)R₂₇, CH₂CN, CH₂N₃, CH₂NH₂, CH₂NHR₂₆, CH₂N(R₂₆)₂, CH₂OH, CH₂OR₂₆, CH₂O(C═O)R₂₇, CH₂O(P═O)(OH)₂, CH₂O(P═O)(OR₂₆)₂, CH₂SH, CH₂S—R₂₆, CH₂SC(═O)R₂₇, CH₂NC(═O)R₂₇, C(═O)CHR₂₈OH, C(═O)CHR₂₈OR₂₆, C(═O)CHR₂₈OC(═O)R₂₇ or
R₁₀ and R₂₅ taken together may be ═C(R₂₈)₂, that is, an optionally alkyl substituted methylene group;
wherein R₂₆═C₁-C₆ (alkyl, branched alkyl, cycloalkyl, haloalkyl, aralkyl, aryl);
R₂₇=R₂₆+OR₂₆; R₂₈=H, C₁-C₆ (alkyl, branched alkyl, cycloalkyl).

2. The method of claim 1 wherein the compound is selected from the group consisting of 21-methyl-5β-pregnan-3α,11β,17α,21-tetrol-20-one 21-methyl ether; 3β-azido-21-acetoxy-5β-pregnan-11β,17α-diol-20-one; 3β-acetamido-21-acetoxy-5β-pregnan-11β,17α-diol-20-one; 5β-pregnan-11β,17α,21-triol-20-one; 4, 9(11)-pregnadien-17α,21-diol-3,20-dione-21-acetate and 4,9(11)-pregnadien-17α,21-diol-3,20-dione.

3. The method of claim 2 wherein the compound is selected from the group consisting of 4,9(11)-pregnadien-17α,21-diol-3,20-dione-21-acetate and 4,9(11)-pregnadien-17α,21-diol-3,20-dione.

4. A composition for treating choroidal neovascularization resulting from ocular surgery or trauma to ocular tissue, said composition comprising a pharmaceutically effective amount of an angiostatic agent having the following structure: 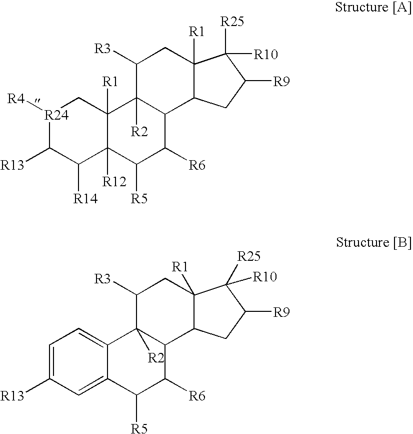
wherein R₁ is H, β-CH₃ or β-C₂H₅;
R₂ is F, C₉-C₁₁ double bond, C₉-C₁₁ epoxy, H or Cl;
R₃ is H, OR₂₆, OC(═O)R₂₇, halogen, C₉-C₁₁ double bond, C₉-C₁₁ epoxy, ═O, —OH, —O-alkyl(C₁-C₁₂),
—OC(═O)alkyl(C₁-C₁₂), —OC(═O)ARYL, —OC(═O)N(R)₂ or
—OC(═O)OR₇, wherein ARYL is furyl, thienyl, pyrrolyl, or pyridyl and each of said moieties is optionally substituted with one or two (C₁-C₄)alkyl groups, or ARYL is —(CH₂)_{f}-phenyl wherein f is 0 to 2 and the phenyl ring is optionally substituted with 1 to 3 groups selected from chlorine, fluorine, bromine, alkyl(C₁-C₃), alkoxy(C₁-C₃), thioalkoxy-(C₁-C₃), Cl₃C—, F₃C—, —NH₂ and —NHCOCH₃ and R is hydrogen, alkyl (C₁-C₄), or phenyl and each R can be the same or different, and R₇ is ARYL as herein defined, or alkyl(C₁-C₁₂);
R₄ is H, CH₃, Cl or F;
R₅ is H, OH, F, Cl, Br, CH₃, phenyl, vinyl or allyl;
R₆ is H or CH₃;
R₉ is CH₂CH₂OR₂₆, CH₂CH₂C(═O)R₂₇, H, OH, CH₃, F, ═CH₂, CH₂C(═O)OR₂₈, OR₂₆, O(C═O)R₂₇ or O(C═O)CH₂(C═O)OR₂₆
R₁₀ is —C≡CH, —CH═CH₂, halogen, CN, N₃, OR₂₆, OC(═O)R₂₇, H, OH, CH₃ or R₁₀ forms a second bond between positions C-16 and C-17;
R₁₂ is H or forms a double bond with R₁ or R₁₄;
R₁₃ is halogen, OR₂₆, OC(═O)R₂₇, NH₂, NHR₂₆, NHC(═O)R₂₇, N(R₂₆)₂, NC(═O)R₂₇, N₃, H, —OH, ═O, —O—P(═O)(OH)₂, or —O—C(═O)—(CH₂)ₜCOOH where t is an integer from 2 to 6;
R₁₄ is H or forms a double bond with R₁₂;
R₁₅ is H, ═O or —OH;
and R₂₃ with R₁₀ forms a cyclic phosphate;
wherein R₉ and R₁₅ have the meaning defined above;
or wherein R₂₃ is —OH, O—C(═O)—R₁₁, —OP(O)—(OH)₂, or —O—C(═O)—(CH₂)ₜCOOH wherein t is an integer from 2 to 6; and R₁₁ is —Y—(CH₂)ₙ—X—(CH₂)ₘ—SO₃H,
—Y′—(CH₂)ₚ—X′—(CH₂)_{q}—NR₁₆R₁₇ or -Z(CH₂)ᵣQ,
wherein Y is a bond or —O—; Y′ is a bond, —O—, or —S—; each of X and X′ is a bond, —CON(R₁₈)—, —N(R₁₈)CO—, —O—, —S—, —S(O)—, or —S(O₂)—; R₁₈ is hydrogen or alkyl (C₁-C₄); each of R₁₆ and R₁₇ is a lower alkyl group of from 1 to 4 carbon atoms optionally substituted with one hydroxyl or R₁₆ and R₁₇ taken together with the nitrogen atom to which each is attached forms a monocyclic heterocycle selected from pyrrolidino, piperidino, morpholino, thiomorpholino, piperazino or N(lower)alkyl-piperazino wherein alkyl has from 1 to 4 carbon atoms; n is an integer of from 4 to 9; m is an integer of from 1 to 5; p is an integer of from 2 to 9; q is an integer of from 1 to 5;
Z is a bond or —O—; r is an integer of from 2 to 9; and Q is one of the following:
(1) —R₁₉—CH₂COOH wherein R₁₉ is —S—, —S(O)—, —S(O)₂—, —SO₂N(R₂₀)—, or N(R₂₀)SO₂—; and R₂₀ is hydrogen or lower allyl-(C₁-C₄); with the proviso that the total number of carbon atoms in R₂₀ and (CH₂)ᵣ is not greater than 10; or
(2) —CO—COOH; or
(3) CON(R₂₁)CH(R₂₂)COOH wherein R₂, is H and R₂₂ is H, CH₃, —CH₂COOH, —CH₂CH₂COOH, —CH₂OH, —CH₂SH, —CH₂CH₂SCH₃, or —CH₂Ph-OH wherein Ph-OH is p-hydroxyphenyl;
or R₂₁ is CH₃ and R₂₂ is H;
or R₂₁ and R₂₂ taken together are —CH₂CH₂CH₂—;
or —N(R₂₁)CH(R₂₂)COOH taken together is —NHCH₂CONHCH₂COOH; and pharmaceutically acceptable salts thereof;
with the proviso that if R₂₃ is a phosphate, it must form a cyclic phosphate, with R₁₀ when R₁₃ is ═O, except for the compound wherein R₁ is β-CH₃, R₂ and R₃ taken together form a double bond between positions 9 and 11, R₄ and R₆ are hydrogen, R₁₂ and R₁₄ taken together form a double bond between positions 4 and 5, R₅ is α-F, R₉ is p-CH₃, R₁₀ is α-OH, R₁₃ and R₁₅ are ═O and R₂₃ is —OP(O)—(OH)₂.
R₂₄═C, C₁-C₂ double bond, O;
R₂₅═C(R₁₅)CH₂—R₂₃, OH, OR₂₆, OC(═O)R₂₇, R₂₆, COOH, C(═O)OR₂₆, CHOHCH₂OH, CHOHCH₂OR₂₆, CHOHCH₂C(═O)R₂₇, CH₂CH₂OH, CH₂CH₂OR₂₆, CH₂CH₂C(═O)R₂₇, CH₂CN, CH₂N₃, CH₂NH₂, CH₂NHR₂₆, CH₂N(R₂₆)₂, CH₂OH, CH₂OR₂₆, CH₂O(C═O)R₂₇, CH₂O(P═O)(OH)₂, CH₂O(P═O)(OR₂₆)₂, CH₂SH, CH₂S—R₂₆, CH₂SC(═O)R₂₇, CH₂NC(═O)R₂₇, C(═O)CHR₂₈OH, C(═O)CHR₂₈OR₂₆, C(═O)CHR₂₈OC(═O)R₂₇ or
R₁₀ and R₂₅ taken together may be ═C(R₂₈)₂, that is, an optionally alkyl substituted methylene group;
wherein R₂₆═C₁-C₆ (alkyl, branched alkyl, cycloalkyl, haloalkyl, aralkyl, aryl);
R₂₇=R₂₆+OR₂₆; R₂₈=H, C₁-C₆ (alkyl, branched alkyl, cycloalkyl).

5. The composition of claim 4 wherein the angiostatic agent is selected from the group consisting of 21-methyl-5β-pregnan-3α,11β,17α,21-tetrol-20-one 21-methyl ether; 3β-azido-21-acetoxy-5β-pregnan-11β,17α-diol-20-one; 3β-acetamido-21-acetoxy-5β-pregnan-11β,17β-diol-20-one; 5α-pregnan-11β,17α,21-triol-20-one; 4,9(11)-pregnadien-17α,21-diol-3,20-dione-21-acetate and 4,9(11)-pregnadien-17α,21-diol-3,20-dione.

6. The composition of claim 4 wherein the compound is present at a concentration between 0.005 and 5.0 weight percent.

7. The composition of claim 5 wherein the compound is 4,9(11)-pregnadien-17α,21-diol-3,20-dione-21-acetate or 4,9(11)-pregnadien-17α,21-diol-3,20-dione.

8. The composition of claim 6 wherein the compound is present at a concentration of between 0.05 and 2.0 weight percent.
